SEQUENCE LISTING (14 sequences)

SEQ ID NO: 1 - DNA, 823 bp, Homo sapiens
  misc_feature (1)...(823): n = A,T,C or G
gcnntgccgcctataattaagnngagaaattaactatgagaggatcgcatcaccatcaccatcacggatcccccgggctgcaggaattcggcacgagggctacttgggaggctgaagtgggaggatggcctgagctcaaggagatgcaggctgcagtgggctgtgattgtgccactgcactccagcctgggcaccaatgtgagcctcgtgccgaattcggcacgagggcggcgttggcggcttgtgcagcaatggccaagatcaaggctcgagatcttcgcgggaagaagaaggaggagctgctgaaacagctggacgacctgaaggtggagctgtcccagctgcgcgtcgccaaagtgacaggcggtgcggcctccaagctctctaagatccgagtcgtccggaaatccattgcccgtgttctcacagttattaaccagactcagaaagaaaacctcaggaaattctacaaggggcaagaagtacaagcccctggaacttgcggcctaagaagacacgtgccatgcgccgccggctcaacaagcaccaagaaaacctgaanaccaagaagcagcaancnggaaggaccggcttgtaacccgcttgcnggaaattacccggtcaaggccntgaggggcgcattggtcaataaaaccacaacctggcntgagaaactcaccccanntntncctnactcgagggggggggcccgggtaanccccggggtttcgaaccttgcaaanccaanctttaatttaacttgaacctttgggaacttccctggttgnattaanntnccaattnaatgaaccnnnaaaaaccc

SEQ ID NO: 2 - protein, 194 aa, Homo sapiens
HHHHHHMAASAFAGAVRAASGILRPLNILASSTYRNCVKNASLISALSTGRFSHIQTPVVSSTPRLTTSERNLTCGHTSVILNRMAPVLPSVLKLPVRSLYYFSARKGKRKTVKAVIDRFLRLHCGLWVRRKAGYKKKLWKKTPARKKRLREFVFCNKTQSKLLDKMTTSFWKRRNWYVDDPYQKYHDRTNLKV

SEQ ID NO: 3 - DNA, 744 bp, Homo sapiens
  misc_feature (1)...(744): n = A,T,C or G
gcnnnnnngcnnnnnnnngggcnccnanaaatagccgatcnacctggngctttttatcgcaactctctactgtttctccatacccgtttnttttgggctanaaataattttgtttaactttaagaaggagatatacatacccatgggctctggatccggtgatgacgatgacaagctcgcccttaaaccctcactaaagggaacaaaagctggagctccaccgcggtggcggccgctctagaactagtggatcccccgggctgcaggaattcggcacgagggtttgccgccagaacacaggtgtcgtgaaaactacccctaaaagccaaaatgggaaaggaaaagactcntatcaacattgtcgtcattggacacgtagattcgggcaagtccaccactactggccgtcgtnttacaagggcgagcttgaaggtaagcctatccctaaccctctcctcggtctcgattctacgcgtaccggtcatcatcaccatcaccattgagtttaaacggtctccancttggctgtttttggcggatgagagaagattttcagcctgatacagattaaaatcagaacgcagaagcggtctgataaaacagaatttgcctggcgggnagtnaccgcgggtgggtccaaccttgaaccccaattgcccgaactcagaaagtgaaaaccgccggtaagccccgaattggttagttgttgggggtcttccccatttgcnaanaagttaggggaaa

SEQ ID NO: 4 - protein, 76 aa, Homo sapiens
  VARIANT (1)...(76): Xaa = Any Amino Acid
TLTKGNKSWSSTAVAAALELVDPPGCRNSARGFAARTQVSZKLPLKAKMGKEKTXINIVVIGHVDSGKSTTTGRRX

SEQ ID NO: 5 - DNA, 542 bp, Homo sapiens
  misc_feature (1)...(542): n = A,T,C or G
gtaaaacgacggccagtgaattgtaatacgactcactatagggcgaattgggtaccgggccccccctcgagtttttttttttttttttattcggctcngtctaatcctttttgtagtcactcataggccagacttngggctagnatgatngattaataagagggatgacataactattagtggncaggntngttgtttgtagnggctcntggcaggggnaaaaggagggcaaatttctagatcaaataaataagaaggtaatagctactaaanaaagaattttaatgnagaaagggacccgggcggnnggatatagggtcnaagccgcnctcgtaaggggtgggatttttctatgtagccnntngagttgtggtnagtcnaaaatttaataaattattagtagtaaaggcctagggagggntgttgccctcgtgcccgaattncctgccagcccggggggaatccncctagttcctaagagccggcccccnccccngaagggangctcccagcctttttgatccctttngtggngngttaat

SEQ ID NO: 6 - protein, 197 aa, Homo sapiens
  VARIANT (1)...(197): Xaa = Any Amino Acid
VKRRPVNCNTTHYRANWVPGPPSSFFFFFYSAXSNPFCSHSZARLXAXMXDZZEGZHNYZWXXXLFVXAXGRXKRRANFZIKZIRRZZLLXKEFZXRKGPGRXDIGXKPXSZGVGFFYVAXXVVXSXKFNKLLVVKAZGGXLPSCPNXLPARGESXZFLRAGPXPXRXAPSLFDPFXXXLIXGGAFKXKAYPXPXPX

SEQ ID NO: 7 - DNA, 705 bp, Homo sapiens
  misc_feature (1)...(705): n = A,T,C or G
attaaccctcactaaagggaacaaaagctggagctccaccgcggtggcgccgctctagaactagtgggatcccccgggctgcaggaaattcggcacgagggaanaatccgncgcgtccacaannaccnttnncccccaaccaacannaanaacanttcnnncnnaaatcnaagtnctccnagactnanaatcnnccatntnatntaaattttcngggggggggnncccngnaancnaaattccccccttanggaagggggnccttntnnanangngnnatnctttaaagncnaaangcctttntncnnnataancccnttntctttgggggctcccnaaattttataaccncnagganccncgggnttctttntttancnccccttnnaaantanttnnnggtnttnaanancggnttcccccncggttntgggcatntntttttncgcgncgnttatagaganaaaaaaaaanttttnttcnccctttatacaccggcanttaaaanttngaaaancngggnaanngggngtttnttnnaaaaaacnaaatnttttntttnagccncnaaaaaaanctgagttggcccccnctnnaaccccnttggngggaaaantnaaaaagtgcaaacccccnctctnccccnatctaganaagtagnntcctccccccctcccnnaaaanntagggagnnnctcccgnnnc

SEQ ID NO: 8 - DNA, 644 bp, Homo sapiens
  misc_feature (1)...(644): n = A,T,C or G
attaaccctcactaaagggaacaaaagctggagctccaccgcggtggcggccgctctagaactagtggatcccccggggngcncgaattcngaangaggcctcntgccnantnctnatganagcgaaggangtannncagntcgnaccngattgaccntnaggatatccantacncnanggggggcccggnncccaatncnccctatagtgagtcnnatcacaattcactggaccgncgtttcaaagggngagntttgggggtaagnctatacctaacccnctctcggnnttganttacacgtncncggtcngtcattcancaancaccaattgagtnttnancnggtcctccaggctngnggttgcntnngggggnnctnagnannaagaattttcaaggctgaaatcccnntttaacccccaantngnnnagnaagggnggtnctgcccaannacaaaaaatttggggatannnggcaaggtnannccangttgnancccaacagggtncccccnngnacagnaacntggggnnatntngaaaacntcnnctnttnncncccnaatngngagtnaatgggggcnnncccccatttggngaaaaattncgngganccggnccncgggantttnaaatnaaanc

SEQ ID NO: 9 - protein, 215 aa, Homo sapiens
  VARIANT (1)...(215): Xaa = Any Amino Acid
INPHZREQKLELHRGGGRSRTSGSPGXXEFXXRPXAXXXZXRRXXXXSXXIDXXDIXYXXGGPXPNXPYSEXXHNSLDXRFKGXXLGVXLYLTXSRXZXTRXRXVIXQXPIEXXXGPPGXXLXXGXXXXKNFQGZNPXLTPXXXXKXGXAQXQKIWGXXARXXXVXXQQGXPXXXNXGXXXKXXXXXPXXXXNGGXPPFXEKXXGXXXRXFXXKX

SEQ ID NO: 10 - DNA, 665 bp, Homo sapiens
  misc_feature (1)...(665): n = A,T,C or G
attaaccctcactaaagggaacaaaagctggagctccaccgcggtggcggccgctctagaactagtggatcccccgggctgcccggtacccaattcgccctatagtgagtcgtattacaattcactggccgtcgttttacaagggcgagcttgaaggtaagcctatccctaaccctctcctcggtctcgattctacgcgtaccggtcatcatcaccatcaccattgagtttaaacggtctccagcttggctgttttggcggatgagagaagattttcagcctgatacagattaaatcagaaacgcangaagngggtcttgataaaaacaagaaatttggccttggcgggnagttagcngcgggtnggtncccaccctngaccccattgcccgaaactcacgnaagntgaaaaccgcccggnaaccgcccgnattgggtaagtggtggggggtccttcccccattgccgaanaagntnnggggaaactngcccagggcacttcaaaatnaaaaaacgnaaaggggctnnangtccgaaaaanaaattgggggcctttcccgggttgnaaacctggttgggtttggggccggggggaacncctcntcctngnagtttnggacaaaaatcccgccnggggnncgcgggattttgaaaccgttntgcnn

SEQ ID NO: 11 - protein, 222 aa, Homo sapiens
  VARIANT (1)...(222): Xaa = Any Amino Acid
INPHZREQKLELHRGGGRSRTSGSPGLPGTQFALZZVVLQFTGRRFTRASLKVSLSLTLSSVSILRVPVIITITIEFKRSPAWLFWRMREDFQPDTDZIRNAXXGSZZKQEIWPWRXVSXGXXPTXDPIARNSXKXKTARXPPXLGKWWGVLPPLPXKXXGNXPRALQNXKTXRGXXSEKXIGGLSRVXNLVGFGAGGNXXSXXFXTKIPXGXRGILKPXCX

SEQ ID NO: 12 - DNA, 661 bp, Homo sapiens
  misc_feature (1)...(661): n = A,T,C or G
taatacgactcactatagggcgaattgggtaccgggccccccctcgagttttttttttttttnnttnttttttnttttnctncttttnttttnttntnnnctcnctttttctatnttctttttncctccactctacnggggnntcccccgnggggcaaaancccnnnnccngggggnnncntntttttttgggggnccccccccngggggggnncccncttttttttttccctttntntggggggtttaaanggggngnttnnnggggnaganattaccnancccccccccccggnnncnnanttcnccgcgantnccggngngtcttcccccctttcccttgnggntttaaagggngccncctnnctttccgnnttttttnngcnnggggaaaaaaaaaaaaatttnnccccctggntncccccaatttnannncccccgncccccccaanaaanggttttnnnnaaanaaanaaaaanttttnctggnggggggcnaaaaaagncggggggggntcccccccccggnnccccctgtgggggtaatttttcaaangggnnaaccccctcntntacccccnnttgttntctggggggggnncccccccnccnctccngaagaaaggngggatanngttcntccctcnacntanaaaaaann

SEQ ID NO: 13 - protein, 16 aa, Homo sapiens
ZYDSLZGELGTGPPLE

SEQ ID NO: 14 - protein, 57 aa, Homo sapiens
LTLTKGNKSWSSTAVAPLNWDPPGCRKFEFPAARGIPLVLERRHRGGAPAFVPFSEG

(19) United States
(12) Patent Application Publication
(10) Pub. No.: US 2008/0131417 A1
(43) Pub. Date: Jun. 5, 2008

(54) MICROARRAY-BASED ANALYSIS OF RHEUMATOID ARTHRITIS MARKERS

(75) Inventor: Stewart J. Lebrun, Irvine, CA (US)

Correspondence Address: KNOBBE MARTENS OLSON & BEAR LLP, 2040 MAIN STREET, FOURTEENTH FLOOR, IRVINE, CA, 92614, US

(73) Assignee: MIRAGENE INC., Anaheim, CA (US)

(21) Appl. No.: 11/852,893

(22) Filed: Sep. 10, 2007

Related U.S. Application Data
(63) Continuation of application No. 10/719,770, filed on Nov. 21, 2003, now abandoned.

(51) Int. Cl. A61K 38/00; C40B 40/10; G01N 33/567; A61P 29/00; A61K 38/43; C40B 30/04

(52) U.S. Cl. 424/94.4; 506/18; 435/7.21; 506/9; 514/12

(57) ABSTRACT

A microarray and method of screening for rheumatoid arthritis (RA) in a mammal is disclosed. The method comprises contacting a sample from the mammal to an immobilized polypeptide or fragment thereof homologous to at least a portion of an RA marker protein, and detecting binding of an antibody from the sample to the immobilized polypeptide or fragment thereof.

### RELATED APPLICATIONS

This application is a continuation of U.S. Ser. No. 10/719,770, filed on Nov. 21, 2003, which claims benefit of priority from U.S. Provisional application No. 60/417,068, filed Oct. 8, 2002, both of which are incorporated herein by reference.

### BACKGROUND OF THE INVENTION

1. Field of the Invention

In one embodiment, the disclosed invention relates to a microarray with markers for rheumatoid arthritis and a method for detection of rheumatoid arthritis. In another embodiment, the invention relates to treatment for the disease.

2. Description of the Related Art

Rheumatoid arthritis (RA) is a chronic systemic disorder that affects joints and surrounding tissues as well as other organ systems. The cause is unknown. Infectious, genetic and hormonal factors have been suggested. RA eventually effects the ability to perform daily activities and overall quality of life.

RA effects both sides of the body equally, most commonly wrists, fingers, knees, feet and ankles. When the synovium (joint lining) is affected, the synovium becomes inflamed, secretes more fluid and the joint becomes swollen. Later, the cartilage becomes rough and pitted and the underlying bone becomes affected. Joint destruction typically begins 1-2 years after the appearance of the disease. Organs may also be affected, particularly the lungs, heart and vascular system.

There is no cure for RA although intervention can delay onset of symptoms. Consequently, an early marker for the disease would be useful to provide an early diagnosis. A rheumatoid factor test is available. However, this test is positive in only about 75% of people with symptoms.

Recent technological advances enable high throughput screening of proteins. These include the sequencing of the human genome and the development of high throughput, robotic screening methods required to handle the numbers of samples involved in a “genome” or “proteome” screen. Characterization of the human genome is clearly the first step towards characterization of the human proteome. The use of the genome to characterize the proteome is commonly referred to as “reverse genomics.”

Mitochondrial dysfunction contributes to cell damage in a number of human diseases. One significant mechanism by which mitochondria damage cells is by producing reactive oxygen species from the respiratory chain. (AugMiesel R, et al, Free Radical Research. 25(2):161-9, 1996.). The studies on synovial fibroblast cultures from patients with rheumatoid or reactive arthritis suggested involvement of mitochondria in the disease process. (Eerola E, et al (1988) Br. J. Rheumatol. (1988) vol 27. Suppl 2:128-31.

A ribosomal protein from the mitochondrial large subunit is described. Antibodies to this protein are expressed at elevated levels in patients suffering from RA. This protein has been identified as the L35 protein of the large (39S) subunit of the mammalian mitochondrial ribosome. It is suggested that this species may serve as a useful marker for RA.

Binding of L35 to killer T-cells (HLA-DR) has been described (Gordon, et al. (1995) Eur. J. Immunol. (1995) vol. 25(5): 1473-1476)). Interference with this interaction may provide relief to persons suffering from RA. Also, autoantibodies against cytoplasmic ribosomes have been implicated in patients suffering from systemic lupus erythematosus (Bonfa, et al. (1987) New England Journal of Medicine vol. 317: pages 265-271). If the L35 protein acts in a similar manner in RA patients, the L35 protein may serve as a possible treatment to relieve disease symptoms. Thus, compositions containing the L35 protein may be useful in the treatment of RA. Other mitochondrial proteins which are useful in the treatment and/or diagnosis of RA are described which include eukaryotic translation elongation factor 1 alpha 2; NADH dehydrogenase 3 (NADH dehydrogenase, subunit 3 (complex I)); Homo sapiens gene for 24-kDa subunit of complex I, exon 7; Homo sapiens mRNA for mitotic kinesin-like protein-1 (MKLP-1 gene); Homo sapiens TBXAS1 gene for thromboxane synthase, exon 2; and Homo sapiens uncoupling protein homolog (UCPH) mRNA.

### SUMMARY OF THE INVENTION

In one embodiment, the present invention is drawn to a protein microarray including mitochondrial proteins as markers of RA. Preferably, the protein microarray includes at least a portion of at least two of the following proteins: L35 protein, eukaryotic translation elongation factor 1 α-2; NADH dehydrogenase 3 (complex I), 24-kDa subunit of complex I, mitotic kinesin-like protein-1, thromboxane synthase, and uncoupling protein homolog. More preferably, the protein microarray includes at least a portion of at least four of the above listed proteins. Even more preferably, the protein microarray includes at least a portion of all of the proteins including L35 protein, eukaryotic translation elongation factor I α-2; NADH dehydrogenase 3 (complex I), 24-kDa subunit of complex I, mitotic kinesin-like protein-1, thromboxane synthase, and uncoupling protein homolog.

In a preferred embodiment, the proteins of the protein microarray are His-tagged. In a preferred embodiment, the proteins of the protein microarray are printed on a charged nickel slide.

In a preferred embodiment, the L35 protein of the protein microarray is represented by a sequence which includes at least a part of the sequence shown in SEQ ID NO: 2.

In a preferred embodiment, the eukaryotic translation elongation factor I α-2 of the protein microarray is represented by a sequence which includes at least a part of the sequence shown in SEQ ID NO: 4.

In a preferred embodiment, the NADH dehydrogenase 3 (Complex I) protein of the protein microarray is represented by a sequence which includes at least a part of the sequence shown in SEQ ID NO: 6.

In a preferred embodiment, the 24-kD subunit of Complex I of the protein microarray is represented by a sequence which includes at least a part of a protein encoded by the sequence shown in SEQ ID NO: 7.

In a preferred embodiment, the mitotic kinesin-like protein-1 of the protein microarray is represented by a sequence which includes at least a part of the sequence shown in SEQ ID NO: 9.

In a preferred embodiment, the thromboxane synthase protein of the protein microarray is represented by a sequence which includes at least a part of the sequence shown in SEQ ID NO: 11.

In a preferred embodiment, the uncoupling protein homolog of the protein microarray is represented by a sequence which includes at least a part of the sequence shown in SEQ ID NO: 13.

In one embodiment, the invention is drawn to a method of screening for rheumatoid arthritis in a mammal including the steps of:
contacting a sample from said mammal to an immobilized polypeptide or fragment thereof homologous to at least a portion of at least one protein selected from the group consisting of L35 protein, eukaryotic translation elongation factor 1 α-2; NADH dehydrogenase 3 (complex I), 24-kDa subunit of complex I, mitotic kinesin-like protein-1, thromboxane synthase, and uncoupling protein homolog; and
detecting binding of an antibody from said sample to said immobilized polypeptide or fragment thereof. In a preferred embodiment, the polypeptide or fragment thereof is immobilized on a microarray. Preferably, the proteins or fragments thereof are His-tagged. Preferably, the proteins or fragments thereof are printed on a charged nickel slide.

In another embodiment, the invention is drawn to a method of treating rheumatoid arthritis in a mammal which includes the steps of administering to said mammal a composition comprising a polypeptide or fragment thereof homologous to at least a portion of at least one protein selected from the group consisting of L35 protein, eukaryotic translation elongation factor 1 α-2; NADH dehydrogenase 3 (complex I), 24-kDa subunit of complex I, mitotic kinesin-like protein-1, thromboxane synthase, and uncoupling protein homolog, said polypeptide or fragment thereof being administered in an amount sufficient to interfere with the binding of an antibody from said mammal.

In another embodiment, the invention is directed to a kit for screening for Rheumatoid Arthritis in a mammal, which includes a mitochondrial marker, homolog or fragment thereof, selected from L35 protein, eukaryotic translation elongation factor 1 α-2, NADH dehydrogenase 3 (complex I), 24-kDa subunit of complex I, mitotic kinesin-like protein-1, thromboxane synthase, or uncoupling protein homolog. Preferably, the mitochondrial marker, homolog or fragment thereof is immobilized on a rigid white substrate. In a preferred embodiment, the mitochondrial marker, homolog or fragment thereof is immobilized on a hydrophobic substrate.

Further aspects, features and advantages of this invention will become apparent from the detailed description of the preferred embodiments which follow.

### BRIEF DESCRIPTION OF THE DRAWINGS

FIG. 1. Summary of the method to produce a large number of over-expression clones for a protein array.

FIG. 2. Example Testing 6×His-tagged Proteins.

FIG. 3. Nickel chip with Proteome library.

FIG. 4. Microarrays showing RA positive tagged proteins. The top panel was screened with serum from control population. The bottom panel was screened with serum from RA population.

FIG. 5 shows three panels. All are spotted with the L35 protein. The upper panel is contacted with anti-His serum. The middles panel with serum from RA patients. The bottom panel is contacted with control serum.

FIGS. 6A-6C relate to a capture assay. FIG. 6A shows a schematic view of the procedure. FIG. 6B illustrates the antibody binding. FIG. 6C shows a P53 capture assay at three different dilutions.

FIGS. 7A-7G relate to a Western Blot-type assay. FIG. 7A shows a schematic view of a Western blot type assay for an auto-antigen panel. FIG. 7B shows a listing of autoimmune disease assay antigens. FIG. 7C shows serum for various autoimmune diseases. FIG. 7D shows a Lupus titration. FIG. 7E shows S.L.E. with corresponding markers. FIG. 7F shows a titration for Sjogrens syndrome. FIG. 7G shows an assay for Sjogrens syndrome.

### DETAILED DESCRIPTION OF THE PREFERRED EMBODIMENT

In one aspect, the present invention relates to a method of producing a microarray that can be used to screen for a disease condition. Preferably, the disease condition is an autoimmune disease. In a preferred embodiment, the disease condition is arthritis, diabetes, Lupus, Multiple sclerosis, Myasthenia gravis, Wegener's granulomatosis or Crohn's disease. More preferably, the disease condition is rheumatoid arthritis.

The microarray can be constructed in a number of ways. In one embodiment, a cDNA library is used to construct a “proteome” library. Each protein produced in the “protein library” can be traced back to a single clone that contains a recombinant human gene derived from the original cDNA library. Hence, each protein is “identified” by reverse genomics (i.e., sequencing of the gene from which it was derived). This allows an investigator to screen many recombinant proteins.

In a preferred embodiment, a cDNA library is obtained and cloned into a His vector such as PQE from Qiagen to create a His library. A suitable host cell is transformed with the cDNA library. The transformed cells are plated on selective media and may be induced to produce protein with a suitable inducer molecule. The colonies are then blotted onto a solid support such as a membrane made of nitrocellulose, nylon or polyvinylidene difluoride (PVDF) or a glass or plastic plate. In a most preferred embodiment, a charged nickel slide is used as a solid support. The colonies are identified using antiHis antibody. The His positive clones may be grown in appropriate media and transferred to a multiwell plate or charged nickel slide. Any 6×His tagged proteins may be bound and tested using the described system.

Ni²⁺ slides can be used to determine disease markers and diseased patients. The His-tagged proteins are printed onto nickel-coated slides, washed and reacted with appropriate serum to identify potential disease markers. Preferably, the serum is from a population afflicted with autoimmune disease. In a preferred embodiment, the autoimmune disease is arthritis, diabetes, Lupus, Multiple sclerosis, Myasthenia gravis, Wegener's granulomatosis or Crohn's disease. More preferably, the autoimmune disease condition is rheumatoid arthritis.

In a preferred embodiment, one pool of serum from a disease population is compared to a pool of serum from a healthy (control) population for determination of possible disease markers. Proteins that react with serum from the diseased population but not from serum from the control population are potential disease markers once false positives have been eliminated. In a preferred embodiment, serum from rheumatoid arthritis patients was reacted with the protein array to identify markers for rheumatoid arthritis.

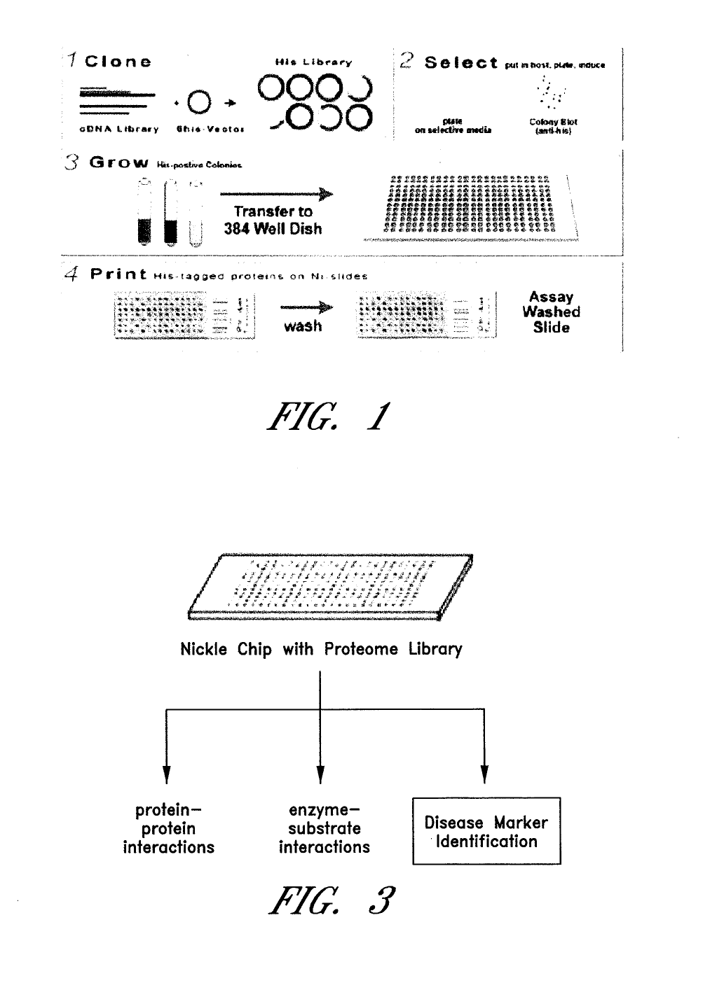

A method is described to produce a large number of over-expression clones for a protein microarray based screening of the “Proteome.” In the Examples that follow, new markers associated with Rheumatoid arthritis as well as other autoimmune diseases were found. FIG. 1 shows the major steps involved in this process.

The positive clone L-35 was obtained by the following steps. Vectors (His-tagged vector from Qiagen:PQE 30, 31, 32) and cDNA libraries were digested with the same restriction enzymes. The digested cDNA libraries were then ligated into the His-tagged vectors. The plasmid clones were transformed into competent cells. Transformants were detected by antibiotic-resistant colony selection. Protein production in the transformants was induced using IPTG. The protein products were screened using serum from rheumatoid arthritis patients. This was done using Ni2+ coated slides which are described below. The protein product was screened with RA and anti-His serum.

The positive clones eukaryotic translation elongation factor 1 alpha 2; NADH dehydrogenase 3/NADH dehydrogenase, subunit 3 (complex I); the human gene for 24-kDa subunit of complex I, exon 7; human mRNA for mitotic kinesin-like protein-1 (MKLP-1 gene); human TBXAS1 gene for thromboxane synthase, exon 2; and human uncoupling protein homolog (UCPH) mRNA were obtained by the following steps. Vectors pBAD-TOPO TA from Invitrogen were used to clone in the PCR product from the cDNA libraries. The selected universal primers were used for amplification. The plasmid clones were transformed into TOP10 competent cells. Transformants were detected by antibiotic-resistant colony selection. Protein production in the transformants was induced with L-arabinose. The protein products were screened using serum from rheumatoid arthritis patients. These were done using charged nickel slides (Z-grip® slides; Miragene Inc.).

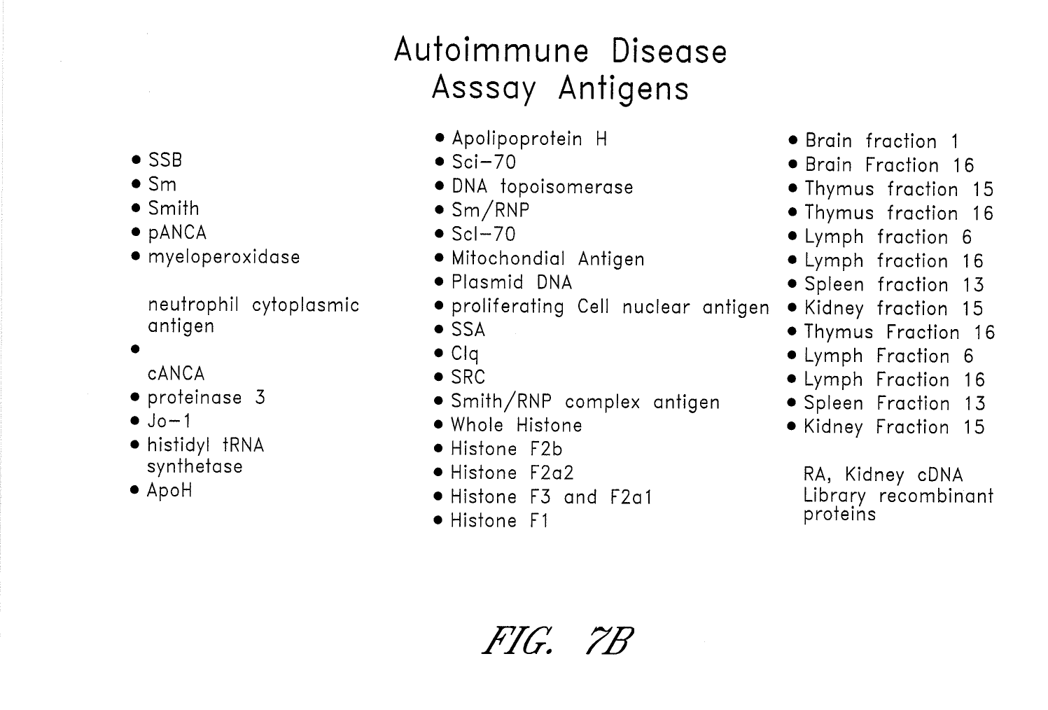

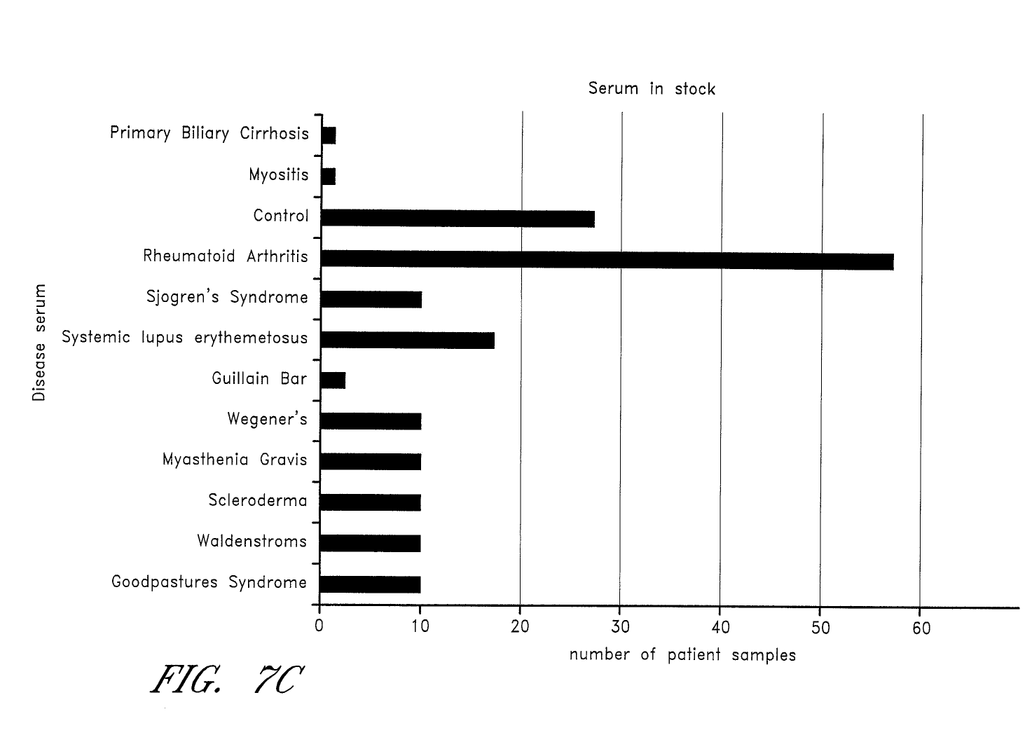

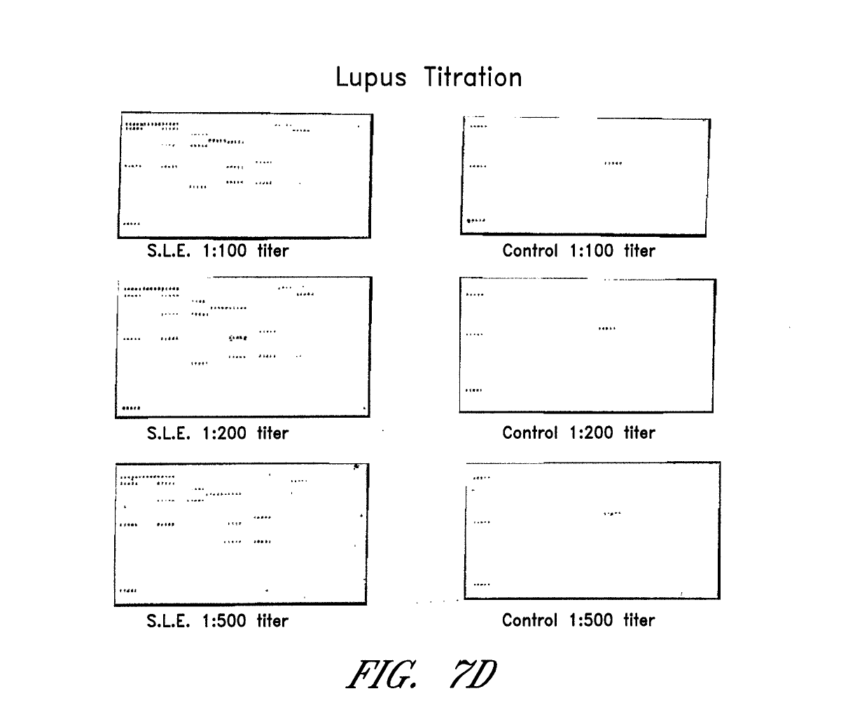

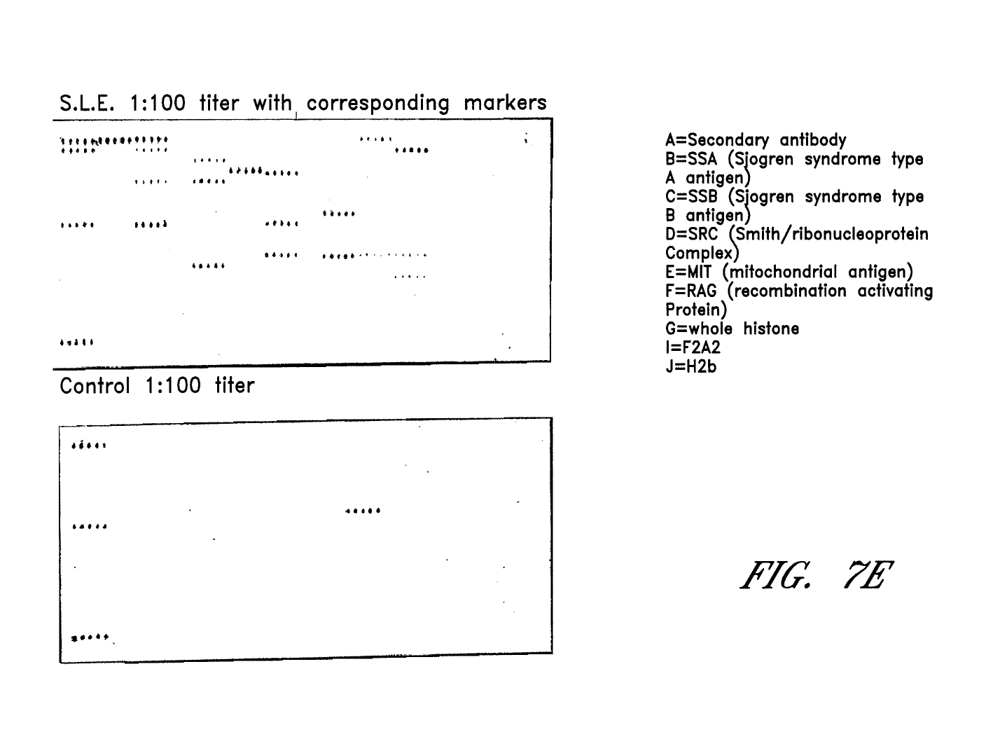

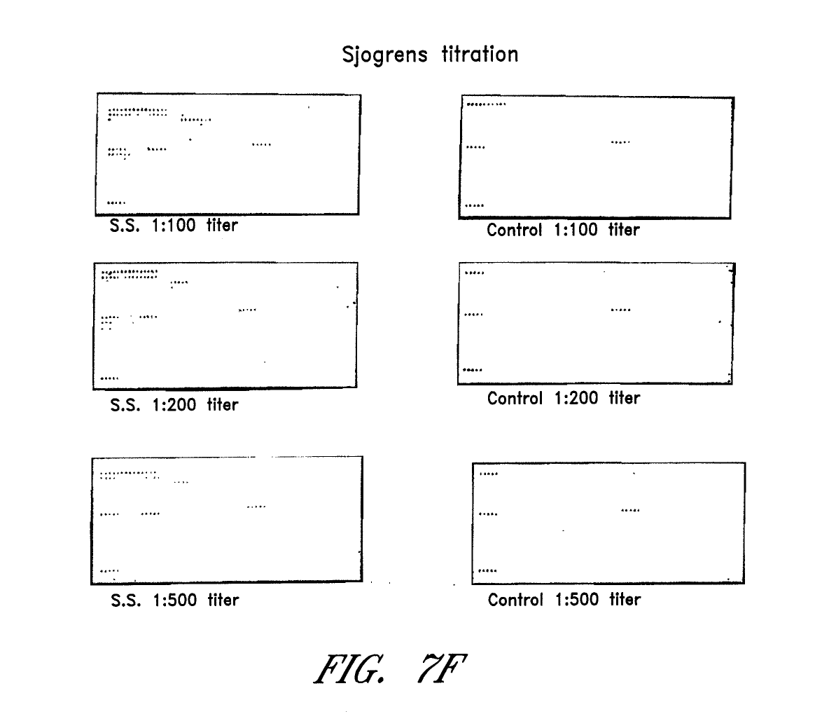

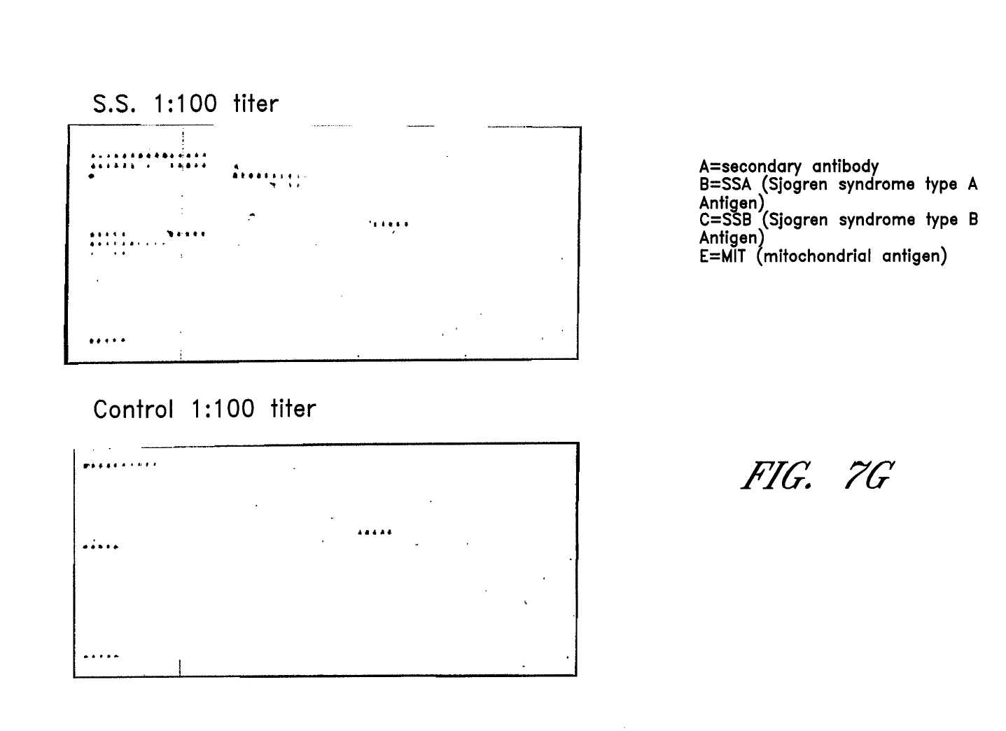

The present invention is not limited to the above RA markers. Additional autoimmune markers have been identified as shown in FIG. 7B. These markers may also be used in the detection and treatment of autoimmune disease.

It will be understood by those of skill in the art that numerous and various modifications can be made without departing from the spirit of the present invention. Therefore, it should be clearly understood that the forms of the present invention are illustrative only and are not intended to limit the scope of the present invention.

### EXAMPLES

### Example 1

### Production of Charged Nickel Slide

A glass surface such as a standard glass slide may be used. Depending upon its application, the glass slide can be chemically etched using hydrofluoric acid to give a white surface rather than a clear one. The first step is to produce amino groups on the glass substrate to allow Ni²⁺ to be coated on the solid surface. This was accomplished by treating clean, dry glass with diluted 3-aminopropyltriethoxysilane (1-4% in dry acetone) for about 15 min, and then rinsing in dry acetone, followed by water. To get the charged nickel onto the surface, the previously treated substrate was soaked in a 20% solution of NiCl₂ for 24 hours, during which the substrate and solution were continuously agitated. After the 24-hour period, the substrate was simply washed away with water (discard the NiCl₂ solution, and agitate the substrate in water for 10 minutes and repeat three times), and air dried over night.

TABLE 1
Formulations:

| Product | Description | Formulation |
|---|---|---|
| Blocker | Casein in TBS | From 10X TBS, dilute to 1X TBS with ddH2O. Add 1 g |
| Fishersci. Cat#37532 | | Casein into 100 ml of 1XTBS = 1% Blocker. Shake |
| | | well and store at 4degree C. |
| IummunoPure Antibody | Anti-goat IgG labeled | |
| Fishersci. Cat#31310 | with Alkaline Phosphate | |
| Developer | 1-Step NBT/BCIP | |
| Fishersci. Cat#34042 | | |
| PBS | 10X | 80 g NaCl, 2 g KCl, 11.5 Na2HPO4•7H2O, 2 g KH2PO4 |
| Fishersci. Cat#175170 | | Mix them into 800 ml of ddH2O, adjust the pH to 7.3 by |
| | | adding NaOH. After measured the pH, add ddH2O up |
| | | to 1000 ml |
| p53 purified protein | | |
| Santa Cruz Biotechnology, | | |
| Cat# sc4246 | | |
| rabbit polyclonal IgG | | |
| Santa Cruz Biotechnology, | | |
| Cat# sc 6243 | | |
| goat anti-rabbit IgG-AP | | |
| Santa Cruz Biotechnology, | | |
| Cat# sc2007 | | |
| mouse monoclonal IgG | | |
| Santa Cruz Biotechnology, | | |
| Cat# sc126 | | |
| abcam | anti-APCN-termius | |
| www.abcam.com | | |

To verify that Ni²⁺ had bound to the glass substrate, a known 6×His tagged protein (w/known density) may be used. In one embodiment, a p 27 purified protein from Santa Cruz Biotechnologies (2161 Delaware Ave., Santa Cruz, Calif. 95060) such as sc4091 was used as a positive control (see Table 1). Self-fabricated proteins (from a RA diseased patient and a control patient) expressing the 6×His-tag were tested to determine if they can be used as disease markers. Two pools of patient serums (one pool of RA patients and one pool of undiseased [control] patients from ProMedDx) may be used. Blocker™—Casein in TBS (Pierce), Human IgG-AP (Immunopure Antibody from Pierce), sc2007—goat anti-rabbit IgG-AP and sc803—his-probe rabbit polyclonal IgG (both from Santa Cruz Biotechnologies), 1×PBS and 1-Step™ NBT/BCIP developer (Pierce) (see Table 1) may be used to develop the results.

To verify that Ni²⁺ had successfully bonded to the glass substrate, and, to determine the sensitivity of the slide 6×His tagged protein (p27) was used. Dilutions of 1:10, 1:100, 1:1000, 1:10000, 1:100000, 1:1000000, 1:10000000, and 1:100000000 μl of p27 to PBS were prepared. These were hand spotted onto the Ni²⁺ slides using a 1-11 pipette. The slide was developed by placing the spotted Ni²⁺ slide into a Petri dish, filling with Blocker and agitating for 30 minutes to 2 hours. The primary antibody was added, in this case, his-probe rabbit polyclonal IgG, with continued shaking for 30 min. to 2 hours. The slide was washed several times with 1×PBS with shaking. The slide was then reacted with secondary antibody, in this case, goat anti-rabbit IgG-AP, in 1×PBS with agitation for 30 minutes to 2 hours. The slide is again washed several times with 1×PBS. Enough Developer was added to cover the slide and shaking was continued until spots began to appear (between 10 and 20-min). The Developer was discarded and enough tap water was added to cover the slide and allowed to sit for a few minutes (1 to 10 minutes) to stop further development. The slides were air-dried overnight. Optionally, the slides may be scanned to get a clearer view of the results. Purple spots indicated that binding between the 6×His and the Ni²⁺ slide had occurred. Sensitivity was determined at the point where the last spot was visible.

The next step verifies that washing is adequate, and that untagged cells do not interfere with the signal. Therefore, control protein was added to each of the 6×His-tagged protein solutions. 1-μl of each of these solutions was then hand spotted onto a Ni²⁺slide, placed in a petri dish, and developed as was described in the previous experiment. The same spots (corresponding to the same dilutions) as the previous experiment should appear. If not, then further washing is necessary, as the cells are interfering and preventing the signal.

### Example 2

### Method of Using Nickel Coated Slides to Test for Disease Markers

Using the prepped 6×His-tagged protein, it is then possible to test whether or not a candidate protein is expressing the 6×His-tag, and to confirm that Elisa tested patients do, in fact, have an autoimmune disease. The tagged proteins were spotted onto three different Ni²⁺ slides (one 1-μl spot of each protein on each slide), where each slide was then placed into its own petri dish. These slides were then developed as described above, with the exception of the added primary and secondary. In one embodiment, the first slide used 40-μl his-probe rabbit polyclonal IgG, as primary and 10-μl goat anti-rabbit IgG-AP as secondary; the second slide used about 10-μl diseased patient serum as primary, and 1-μl human IgG-AP as secondary; the third slide used about 10-μl control serum as primary, and 1-μl human IgG-AP as secondary. Any spots that appeared using the rabbit as primary and goat as secondary indicated that that particular protein was expressing the 6×His-tag. Any of those spots that also appeared on the second slide (when using the diseased patient serum as primary) confirmed that at least one patient from the pool does have the autoimmune disease. And any of those spots that also appeared on the third slide (when using control as primary) indicated that the protein could be a potential disease marker. Note that spotting can also be done by aliquotting about 40-μl of each protein into a well of a 384-wells dish, and using a “Spotbot” to spot onto the Ni²⁺ slides.

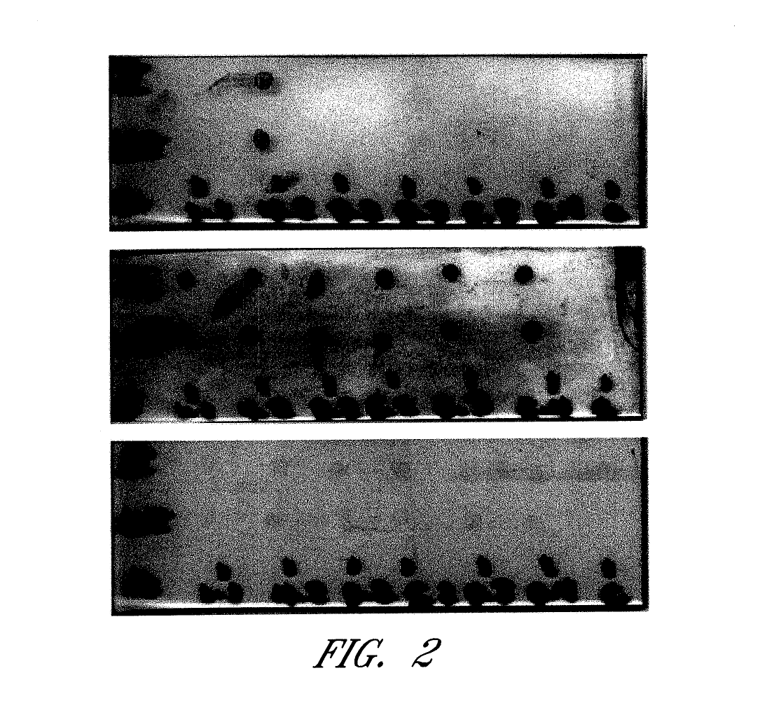

One embodiment is shown in FIG. 2. The top slide (hereafter referred to as “slide A”) consists of six different proteins, developed with 40-μl his probe rabbit polyclonal IgG as primary and 10-μl of goat anti-rabbit IgG-AP as secondary. The middle slide (now referred to as “slide B”) consists of the same six proteins developed with 100-μl of a pool of ten rheumatoid arthritis (RA) patients' serum as primary and 1-μl of human IgG-AP as secondary. The last slide (referred to as slide C) consists of the same six proteins developed with 100-μl of ten control patients' serum as primary, and 1-μl of human IgG-AP as secondary. “I” and “UI” refer to “induced” and “uninduced” respectively.

From slide A (top panel), it can be seen that protein 4.2 gives a strong signal, indicating that this protein is expressing the 6×His-tag. The same protein also gives a strong signal on slide B (middle panel), but a very weak signal on slide C (bottom panel), indicating that the protein is binding to diseased antibodies only. This protein could be a potential disease marker for RA. The appearance of spots on slide b for proteins 1.1, 3.2, 5.2, 5.3, and 5.4, but none on slide A indicates false signals.

### Example 3

### Growing and Maintaining the Host Strains

An aliquot of approximately 50-ul from the host cDNA library strains (an amplified pre-made library constructed in the Uni-Zap XR vector, Quiagen Inc.) was streaked onto LB agar plates (3% Lb etc) containing the appropriate antibiotics for approximately 14-hrs at 37 C. Plates were then sealed with Parafilm and stored at 4° C. for up to 1 week. Cells from the plates were restreaked onto fresh plates every week.

### Example 4

### In Vivo Excision of the pBluescript Phagemid from the Uni-Zap Vector

### Mass Excision Protocol

After cultures of XL1-Blue MRF′ and SOLR cells were grown separately overnight in LB broth supplement with 0.2% (w/v) maltose and 10 mM MgSO₄ at 30° C., the cells were collected (1000×g, sorvall XYZ centrifuge) and re-suspended separately in 10-mM MgSO₄ to an OD 600 of 1.0 (8×10⁸ cells/ml). In a 50-ml conical tube, a portion of the amplified bacteriophage library with XL1-Blue MRF′ (Qiagen, Inc) cells at MOI of 1:10 lambda phage to cell ratio was combined and then ExAssist lambda phage was added at a ratio of 10:1 helper phage to cells. To ensure that every cell was co-infected with lambda phage and helper phage, incubate at 37° C. for 15 min to allow for absorption. To this suspension, 20-ml of LB broth was added to the tube and incubated 2.5-3 hours with shaking (XYZ-rpm). After incubation, debris was removed by centrifugation (Sorvall centrifuge 1000×g for 10-min) the supernatant was collected into the sterile conical tube. 1-μl of this supernatant with 200 μl of SOLR cells described above was combined in a 1.5-ml microcentrifuge tube and incubated at 37° C. for 15-min. 100-μl was plated onto LB-ampicillin agar plate and incubated at 37° C. overnight, of which colonies may be selected for plasmid preparation. Otherwise, instead of plating, 100-μl into LB-ampicillin media can be inoculated and incubated at 37° C. overnight, and then the culture can be used for plasmid preparation to obtain cDNA libraries.

### Example 5

### Isolation of Plasmid DNA from Bacterial Colonies by QIAprep Spin Miniprep

The QIAprep miniprep procedure is based on alkaline lysis of bacterial cells followed by adsorption of DNA onto silica in the presence of high salt. The procedure consists of three basic steps—preparation and clearing of a bacterial lysate; adsorption of DNA onto the QIAprep membrane; washing and elution of plasmid DNA. After cDNA library cultures were inoculated into media, the cultures were incubated overnight in a 37° C. incubator with shaking. 1.5-ml of each culture was transferred into a 1.5 ml micro-centrifuge tube and centrifuged at maximum speed for 1-min in a tabletop micro-centrifuge. The supernatant was removed by aspiration. The bacterial cell pellets were re-suspended in 250-ul of Buffer P1 (Re-suspension buffer: 50-mM Tris-Cl, 10 mM EDTA, 100 ug/ml RNaseA) by pipetting up and down. 250-ul of Buffer P2 (Lysis buffer: 200 mM NaOH, 1% SDS) was added. The samples were mixed gently by inverting the tube 4-6 times because vortexing would result in shearing of genomic DNA. If necessary, the samples were continually mixed until the solution became viscous and slightly clear. The lysis reaction was not allowed to proceed for more than 5 minutes. Then, 350-ul of Buffer N3 (Neutralization buffer: 3.0 M potassium acetate, pH 5.5) was added and the samples were immediately mixed by gently inverting the tube 4-6 times to avoid localized precipitation. The samples were centrifuged for 10 minutes at maximum speed. During the centrifugation, QIAprep spin columns were placed into 2 ml collection tubes. After the centrifugation, a compact white pellet was formed. The supernatant was transferred to the QIAprep column by pipetting. The samples were centrifuged at maximum speed for 1 minute and the flow-through was discarded. To wash the QIAprep spin column, 0.75 ml of Buffer PE (Wash buffer: 96-100% ethanol added) was added. The samples were centrifuged for 30-60 seconds at maximum speed and the flow-through was discarded. The samples were then centrifuged for an additional 1-min to remove residual wash buffer (residual ethanol from Buffer PE may inhibit subsequent enzymatic reactions). QIAprep columns were placed in clean 1.5 ml microcentrifuge tubes. To elute DNA, 50-ul of Buffer EB (10 mM Tris-Cl, pH8.5) was added to the center of each QIAprep column and the column was incubated at room temperature for 1-min. Samples were centrifuged at maximum speed for 1-min. Approximately 50-ul of DNA in EB Buffer was obtained and the concentration was determined by UV spectrophotometry.

### Example 6

### PCR Amplification of Plasmid DNA (for TOPO TA Cloning)

The purified plasmid DNA from Unizap Libraries was used as template for amplification.

The PCR primers were designed or selected without adding of 5′ phosphates to the primers. This allowed the PCR product to be cloned into pBAD TOPO which have 3′-adenine overhang. Universal primers used included T3, T7, M13 forward, and M 13 Reverse primer. Primers were obtained from Operon. PCR was performed by Laragen, Inc. Los Angeles.

### Example 7

### Vectors Preparation (Qiagen, 2001)

The vectors from the QIAexpress Kit (PQE 30, PQE 31, PQE32) were prepared by first dissolving the vectors in TE buffer. A 1 μg aliquot of each then was linearized using the appropriate restriction enzymes. In this project, BamH I and Kpn I were used. The digestions were carried out separately with a clean-up step in between. After restriction digestion, the vector ends were dephosphorylated and ready to be inserted.

### Example 8

### Double Digestion of cDNA libraries and PQE vectors with BamH I/KpnI,

cDNA libraries in pBluescript plasmids were digested with BamH I/Kpn I restriction enzymes. These restriction digests resulted in fragments of pBluescript vector of about 3000 bp and inserted cDNA libraries sized from 0-10,000-bp. The digestion of PQE vectors with the same enzymes resulted in linearization of the vectors at the multiple cloning sites. The samples were separated by electrophoresis on a 1% agarose gel. The restriction digest reactions were set-up as follows in separate 1.5-ml microcentrifuges tubes:

| | | |
|---|---|---|
| Plasmid DNA | 2 | ug |
| 10X Reaction Buffer | 2 | ul |
| Restriction Enzymes | 0.5 | ul (each) |
| 10X BSA | 2 | ul |
| ddH₂O to Total Volume | 20 | ul |

The restriction digest reactions were mixed well by vortexing. The samples were centrifuged at maximum speed to remove drops from the inside of the lid and were then incubated at 37° C. for 2 hours in a water bath.

After incubation, the digested samples were electrophoresed at 100 V for 1 hour on a 1% agarose gel to separate the resulting fragments. After electrophoresis, the selected fragments (0-10,000 kb minus the vectors at about 3000 kb) were excised from the gel. The DNA fragments were extracted from the agarose gel using a QIAquick Gel Extraction Kit, which can only purify DNA fragments up to 10 kb in size.

### Example 9

### Purification of the DNA Fragment from Restriction Enzyme Digestion Using a QIAquick Gel Extraction Kit

This protocol is designed to extract and purify DNA of 70-bp to 10-kb from standard or low-melt agarose gels in TAE or TBE buffer. DNA adsorbs to the silica-membrane in the presence of high salt while contaminants pass through the column. Impurities are efficiently washed away, and the pure DNA is eluted with Tris buffer.

First, a 1.5-ml microcentrifuge tube was weighed before obtaining the gel slice containing the DNA fragment of interest. The DNA fragment was excised from the agarose gel with a clean sharp scalpel, and extra agarose was removed to minimize the size of the gel slice. The gel slice was transferred into a 1.5 ml microcentrifuge tube and weighed. 3 volumes of Buffer QG was added to 1 volume of gel (100 mg˜100 ul) to solubilize the agarose gel slice and to provide the appropriate conditions for binding of DNA to the silica membrane. The gel slice in Buffer QG was incubated at 50° C. for 10 minutes (or until the gel slice had completely dissolved). To help dissolve the gel, the microcentrifuge tube was vortexed to mix the sample every 2-3 minutes during the incubation. After the gel slice had dissolved completely, the mixture should be yellow in color similar to Buffer QG without the dissolved agarose. If the color of the mixture was orange or violet, 10-ul of 3M sodium acetate, pH 5.0 was added and the color of the mixture then turned yellow. 1 gel volume of isopropanol was added and the sample was mixed thoroughly. This step increases the yield of DNA fragments <500 bp and >4 bp. Next, a QIAquick spin column was placed in a 2 ml collection tube. To bind DNA, the sample was applied to the QIAquick column and the column was centrifuged for 1 minute at maximum speed. After centrifugation, the flow-through was discarded. The QIAquick column was placed back in the same collection tube. To wash, 0.75 ml of Buffer PE (96-100% ethanol added) was added to the QIAquick column and the sample was centrifuged for 1 minute at maximum speed. The flow-through was again discarded. The QIAquick column was centrifuged for an additional 1 minute at maximum speed and then placed into a clean 1.5 ml microcentrifuge tube. To elute DNA, 30 ul of Buffer EB (10 mM Tris-Cl, pH8.5) was added to the center of the QIAquick membrane and the column was incubated for 1 minute at room temperature. The column was centrifuged at maximum speed for 1 minute, and approximately 30 ul of DNA sample was obtained.

### Example 10

### Ligation of cDNA Libraries and PQE Vectors (Fisher, 01)

The insert prepared from cDNA libraries was inserted to the vectors. As a negative control, the vector was ligated to itself. The negative control determined how effectively the vectors were dephosphorylated. The starting vector:insert ratio when cloning into a plasmid vector were 1:1, 1:3 or 3:1 molar ratio. The following reaction used 1:1 vectors:insert ratio. Typical ligation reaction used 100-200 ng of vector DNA. The following reaction was assembled in the microcentrifuge tube.

| | | |
|---|---|---|
| Vector DNA | 100 | ng |
| Inserted DNA | 17 | ng |
| Ligase 10X buffer | 1 | μl |
| T4 DNA ligase (weiss unit) | 0.1-1 | u |
| Nuclease-free water to the final volume | 10 | μl |

Then the reaction was incubated at room temperature for 3 hours or at 4 C overnight and was ready to be transformed.

### Example 11

### Ligation of cDNA Libraries and TOPO-TA Vectors and Transformation of Ligation Product into JM 109 Competent Cells or TOP 10 competent Cells by the Heat-Shock Procedure

TOPO cloning reaction using pBAD-TOPO (Invitrogen,) was performed according to the manufacturer's instructions. After ligation, the samples were transformed into JM 109 or TOP 10 competent cells as selected. Before the transformation, competent cells were gently thawed on ice. A 200 ul aliquot of the cells was transferred into pre-chilled Falcon 2059 polypropylene tubes. 1 ul of the ligation samples was then added into 200 ul of the competent cells. The transformation reactions were gently mixed by swirling and incubated on ice for 45-60 minutes (5-30 min for TOP 10 cells). After incubation, the samples were heat shocked for 90 seconds at 42° C. in a water bath and immediately placed on ice for 2 minutes. 0.9 ml of room temperature S.O.C medium (0.2% bactotryptone, 0.06% yeast extract, 1 mM NaCl, 0.25 mM KCl, 1 mM MgCl₂, 1 mM MgSO₄, 2 mM Glucose) was added to the tube. The reaction tube was incubated in a 30° C. incubator to prevent potential DNA degrading enzymes from acting upon the unstable plasmid. The incubation was carried out for 90 minutes (60 minutes for TOP 10 competent cells) with shaking at 225 rpm. 200 ul, 300 ul and 500 ul of transformation reaction were plated on LB-Amp (100 ug/ml Amp) plates. The plates were incubated overnight at 30° C.

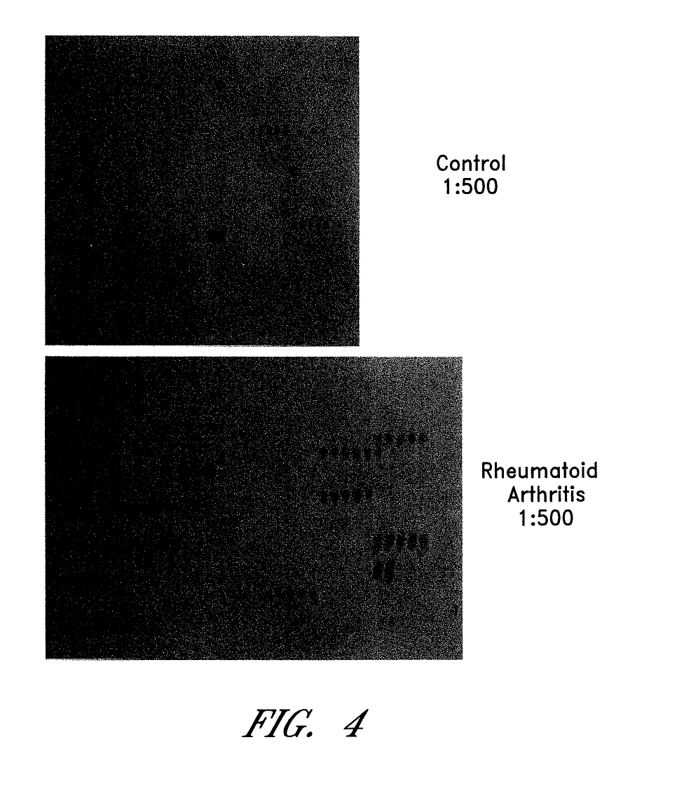

Protein production in the transformants was induced. IPTG was used for inducing the clones using PQE vectors and L-Arabinose was used for the clones using pBAD as vectors. The protein products were screened using serum from rheumatoid arthritis (RA) patients. This was done using Ni²⁺ coated slides as described below (FIG. 3). The protein product was screened with RA and anti-His serum.

### Example 12

### Spotting of 6×His-Tagged Proteins onto Nickel Slides

Spotting of the 6×His-tagged proteins onto the Ni²⁺ slide can be achieved by hand spotting, or by using a commercially available device such as the SpotBot® device (SpotBot®, TeleChem Int., Inc.).

Using hand spotting, a pipette was used to drop 1 μL of the 6×His-tagged protein onto a Ni²⁺ slide, taking note of what the protein is and where on the slide it is spotted. Spotting of 6×His-tagged proteins with SpotBot® was performed as follows. The wash buffer reservoir is filed with a wash buffer (TeleChem International, Inc.) and connected to a wash water container. A peristaltic pump is activated and run for about 5 minutes. Small (40 μl) aliquots of the 6×His-tagged proteins were transferred into the wells of a 384-well dish, and the dish was placed on the left side of the Spotbot®, noting which protein is in which well, as a reference. Ni²⁺ slides were fitted on the right side of the Spotbot with plain microscope slides placed in the pre-print area. The software program “SPOCLE Generator” was used and the spotting procedure was performed according to the SPOTBOT® manual.

### Example 13

### Assay for Detection of Antibodies to 6×His-Tagged Proteins Using Spotbot®

After printing, the preprint slides were discarded and the Ni²⁺ slides were labeled. Each slide was placed in a petri dish with 10 ml of Blocker (Blocker® Casein in TBS (Pierce)). The slides were incubated with shaking for about 1 hour. About 20 μl of Serum (primary antibody) from either the disease (RA) group or the control group was transferred directly into the Blocker® solution. After further incubation for about 1 hour, the slides were washed three times with wash buffer (20 mM imidazole in PBS) and shaking for about 10 minutes.

10 ml of wash buffer and 1 μl of goat anti-human IgG/AP (Goat anti-human IgG labeled with Alkaline Phosphatase ImmunoPure® Antibody (Pierce)) were added to the wash solution. Again the slides were washed three times with wash buffer and shaking. 10 ml of Developer solution (1-Step™ NBT/BCIP (Pierce)) was added to each petri dish and incubated with shaking until spots began to appear (about 10-30 min.). Development was stopped with tap water. The slides were air-dried overnight. The slides were scanned using an EPSON PERFECTION 1650 scanner. 1600 dpi was used for scanning slides. All other scanner settings were factory default settings. Adobe Photoshop 6.0 was used to analyze the scanned files.

### Example 14

### Results

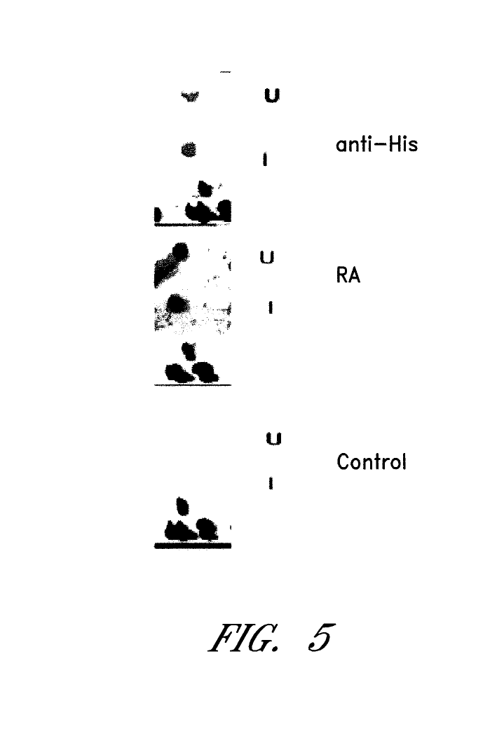

FIG. 4 shows the results of a screen for RA markers. The image on the bottom panel of FIG. 4 used rheumatoid arthritis patient serum as the primary antibody, where the image on the upper panel of FIG. 4 used control patient serum. As can be seen, there are 12 sets of spots using the patient serum, vs. 3 sets of spots using the control serum. On the control, only 2 sets show all 5 spots. This indicates that 2 different his tagged proteins are false positives, and 10 different proteins are rheumatoid arthritis positive.

For one of the positive results described above, the corresponding clone was located. This clone was amplified by inoculation into growth media. The recombinant plasmid was isolated, digested with restriction enzymes and size was determined by Agarose gel electrophoresis. The clone was sequenced using standard procedures. The DNA sequence is set forth below (Table 2; SEQ ID NO: 1). Comparison with available NCBI databases indicated that the isolated sequence encodes a protein of the large subunit of the human mitochondrial ribosome, the L35 protein (Koc, et al. (Nov. 23, 2001) The Journal of Biological Chemistry vol. 276 (47): 43958-43969).

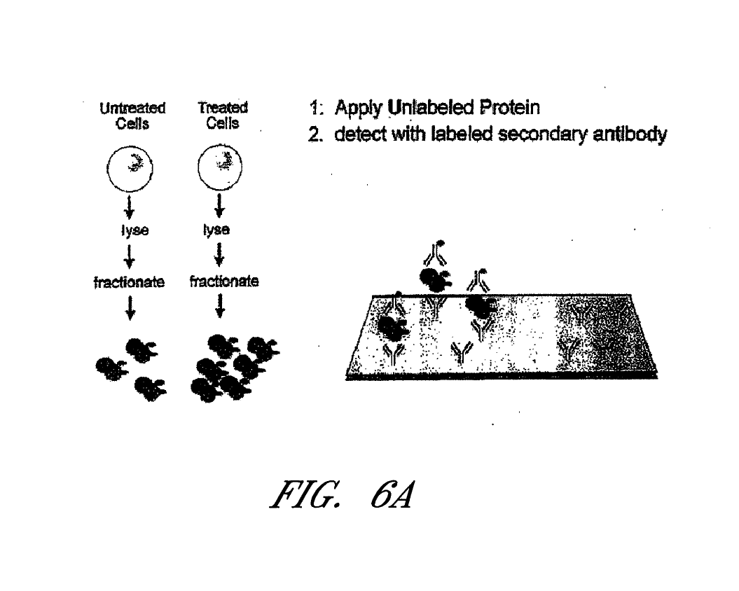

FIG. 5 confirms that L35 is a marker for RA. FIG. 5 shows nickel coated slides that have been prepared as described above and spotted with L35 protein. The upper panel is then contacted with the anti-His serum to confirm the presence of a recombinant protein. The middle panel is contacted with serum from RA patients. The bottom panel is contacted with serum from a control population. It can be clearly seen that the L35 protein only reacts with the anti-His and RA serum, confirming that this protein is a marker for RA. There was no difference in expression between samples induced with IPTG and uninduced.

By the methods described above, a number of markers for RA have now been identified. These are:

Eukaryotic translation elongation factor 1 alpha 2 is encoded by the polynucleotide of SEQ ID NO: 3 (Table 4).

NADH dehydrogenase 3; NADH dehydrogenase, subunit 3 (complex I) is encoded by the polynucleotide of SEQ ID NO: 5 (TABLE 6).

Homo sapiens gene for 24-kDa subunit of complex I, exon 7 is encoded by the polynucleotide of SEQ ID NO: 7 (TABLE 8).

Homo sapiens mRNA for mitotic kinesin-like protein-1 (MKLP-1 gene) is encoded by the polynucleotide of SEQ ID NO: 8 (TABLE 10).

Homo sapiens TBXAS1 gene for thromboxane synthase, exon 2. is encoded by the polynucleotide of SEQ ID NO: 10 (TABLE 12).

Human uncoupling protein homolog (UCPH) mRNA is encoded by the polynucleotide of SEQ ID NO: 12 (TABLE 14).

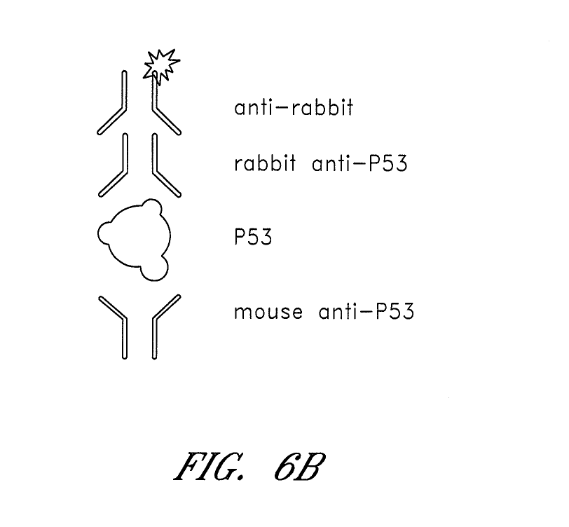

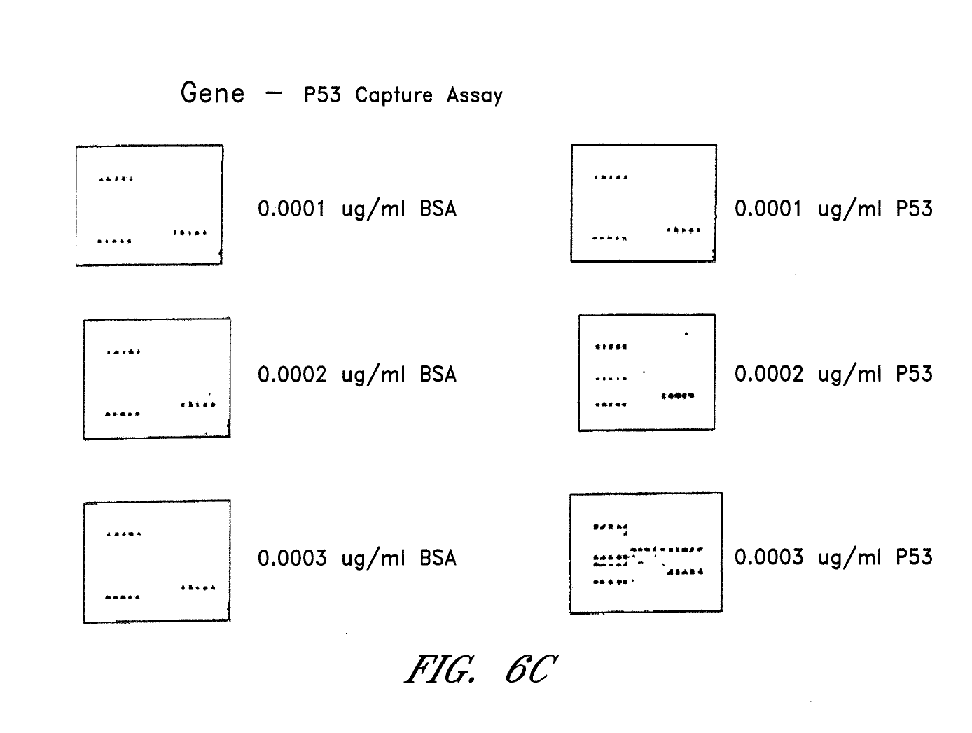

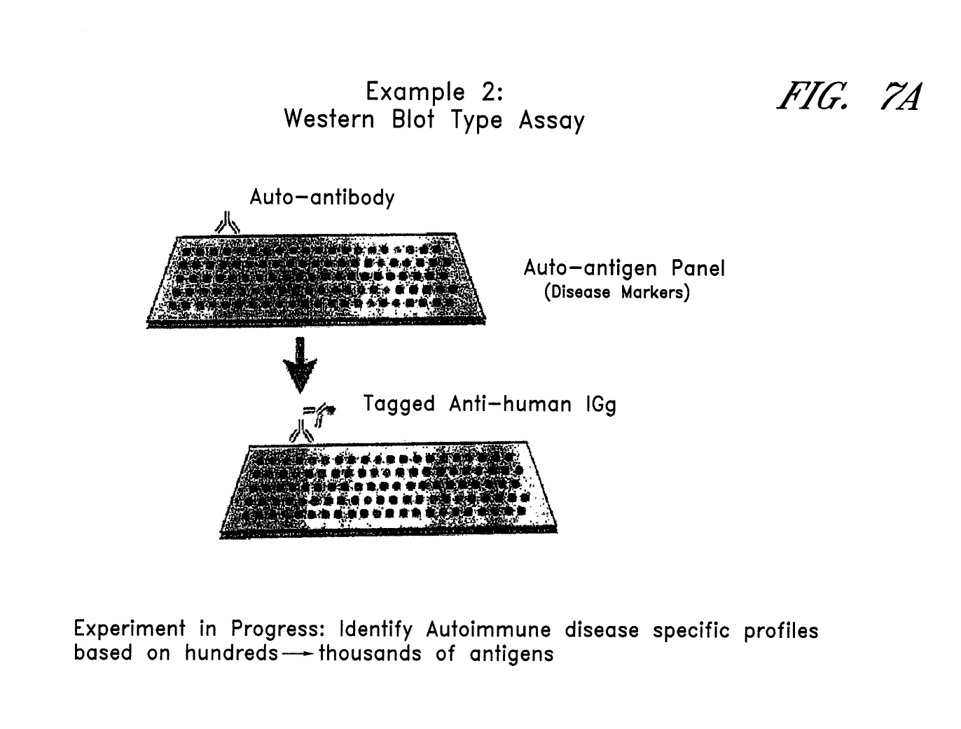

Alternate methods of protein screening may also be used. In a method which is substantially similar to the method described by Chin et al. in U.S. Pat. No. 6,197,599, which is incorporated herein by reference (Appendix A), antibodies are attached in a microarray as shown in FIG. 6A. The antibody-treated surface is contacted with an unlabeled protein preparation. Detection is carried out with a labeled secondary antibody. See FIG. 6B which shows isolation and identification of the p53 protein. The p53 tumor-suppressor protein has been implicated in RA and overexpression of p53 is a characteristic feature of the disease (Sun et al. (April 2002) Semin Arthritis Rheum vol. 31 (5):299-310). FIG. 6C shows the sensitivity of the assay.

FIG. 7A shows another variation where a solid substrate presents an array of disease markers. Identification is carried out by treatment with auto-antibody. FIG. 7B presents some known autoimmune disease assay antigens and FIG. 7C shows graphically the number of patients in each disease population. FIGS. 7D-G show the feasibility of the method for other disease markers.

TABLE 2
The DNA sequence of L35 (SEQ ID NO: 1).

GCNNTGCCGCCTATAATTAAGNNGAGAAATTAACTATGAGAGGATCGCAT
CACCATCACCATCACGGATCCCCCGGGCTGCAGGAATTCGGCACGAGGGC
TACTTGGGAGGCTGAAGTGGGAGGATGGCCTGAGCTCAAGGAGATGCAGG
CTGCAGTGGGCTGTGATTGTGCCACTGCACTCCAGCCTGGGCACCAATGT
GAGCCTCGTGCCGAATTCGGCACGAGGGCGGCGTTGGCGGCTTGTGCAGC
AATGGCCAAGATCAAGGCTCGAGATCTTCGCGGGAAGAAGAAGGAGGAGC
TGCTGAAACAGCTGGACGACCTGAAGGTGGAGCTGTCCCAGCTGCGCGTC
GCCAAAGTGACAGGCGGTGCGGCCTCCAAGCTCTCTAAGATCCGAGTCGT
CCGGAAATCCATTGCCCGTGTTCTCACAGTTATTAACCAGACTCAGAAAG
AAAACCTCAGGAAATTCTACAAGGGGCAAGAAGTACAAGCCCCTGGAACT
TGCGGCCTAAGAAGACACGTGCCATGCGCCGCCGGCTCAACAAGCACCAA
GAAAACCTGAANACCAAGAAGCAGCAANCNGGAAGGACCGGCTTGTAACC
CGCTTGCNGGAAATTACCCGGTCAAGGCCNTGAGGGGCGCATTGGTCAAT
AAAACCACAACCTGGCNTGAGAAACTCACCCCANNTNTNCCTNACTCGAG
GGGGGGGGCCCGGGTAANCCCCGGGGTTTCGAACCTTGCAAANCCAANCT
TTAATTTAACTTGAACCTTTGGGAACTTCCCTGGTTGNATTAANNTNCCA
ATTNAATGAACCNNNAAAAACCC

Table 3 shows the protein sequence for the L35 protein (SEQ ID NO: 2) identified and isolated as described herein. The first six residues represent the 6×His tag.

TABLE 3

HHHHHHMAASAFAGAVRAASGILRPLNILASSTYRNCVKNASLISALSTG
FRSHIQTPVVSSTPRLTTSERNLTCGHTSVILNRMAPVLPSVLKLPVSLY
YFSARKGKRKTVKAVIDRFLRLHCGLWVRRKAGYKKKLWKKTPARKKRLR
EFCNKTQSKLLDKMTTSFWKRRNWYVDDPYQKYHDRTNLKV

TABLE 4

The DNA sequence for eukaryotic translation factor
1 alpha (SEQ ID NO: 3).

GCNNNNNNGCNNNNNNNNGGGCNCCNANAAATAGCCGATCNACCTGGNGC
TTTTTATCGCAACTCTCTACTGTTTCTCCATACCCGTTTNTTTTGGGCTA
NAAATAATTTTGTTTAACTTTAAGAAGGAGATATACATACCCATGGGCTC
TGGATCCGGTGATGACGATGACAAGCTCGCCCTTAAACCCTCACTAAAGG
GAACAAAAGCTGGAGCTCCACGCGGTGGCGGCCGCTCTAGAACGTAGTGG
ATCCCCCGGGCTGCAGGAATTCGGCACGAGGGTTTGCCGCCAGAACACAG
GTGTCGTGAAAACTACCCCTAAAAGCCAAAATGGGAAAGGAAAAGACTCN
TATCAACATTGTCGTCATTGGACACGTAGATTCGGGCAAGTCCACCACTA
CTGGCCGTCGTNTTACAAGGGCGAGCTTGAAGGTAAGCCTATCCCTAACC
CTCTCCTCGGTCTCGATTCTACGCGTACCGGTCATCATCACCATCACCAT
TGAGTTTAAACGGTCTCCANCTTGGCTGTTTTTGGCGGATGAGAGAAGAT
TTTCAGCCTGATACAGATTAAAATCAGAACGCAGAAGCGGTCTGATAAAA
CAGAATTTGCCTGGCGGGNAGTNACCGCGGGTGGGTCCAACCTTGAACCC
CAATTGCCCGAACTCAGAAAGTGAAAACCGCCGGTAAGCCCCGAATTGGT
TAGTTGTTGGGGGTCTTCCCCATTTGCNAANAAGTTAGGGGAAA

TABLE 5

shows the protein sequence for part of eukaryotic
translation factor 1 alpha 2 (SEQ ID NO: 4).

TLTKGNKSWSSTAVAAALELVDPPGCRNSARGFAARTQVSZKLPLKAKMG
KEKTXINIVVIGHVDSGKSTTTGRRX

TABLE 6

The DNA sequence of NADH dehydrogenase 3
(SEQ ID NO: 5)

GTAAAACGACGGCCAGTGAATTGTAATACGACTCACTATAGGGCGAATTG
GGTACCGGGCCCCCCCGAGTTTTTTTTTTTTTTTTTATTCGGCTCNGTCT
AATCCTTTTTGTAGTCACTCATAGGCCAGACTTNGGGCTAGNATGATNGA
TTAATAAGAGGGATGACATAACTATTAGTGGNCAGGNTNGTTGTTTGTAG
NGGCTCNTGGCAGGGGNAAAAGGAGGGCAAATTTCTAGATCAAATAAATA
AGAAGGTAATAGCTACTAAANAAAGAATTTTAATGNAGAAAGGGACCCGG
GCGGNNGGATATAGGGTCNAAGCCGCNCTCGTAAGGGGTGGGATTTTTCT
ATGTAGCCNNTNGAGTTGTGGTNAGTCNAAAATTTAATAAATTATTAGTA
GTAAAGGCCTAGGGAGGGNTGTTGCCCTCGTGCCCGAATTNCCTGCCAGC
CCGGGGGGAATCCNCCTAGTTCCTAAGAGCCGGCCCCCNCCCCNGAAGGG
ANGCTCCCAGCCTTTTTGATCCCTTTNGTGGNGNGTTAAT

TABLE 7

shows the protein sequence for part of NADH
dehydrogenase 3; NADH dehydrogenase, subunit 3
(complex I) (SEQ ID NO: 6).

VKRRPVNCNTTHYRANWVPPPSSFFFFFYSAXSNPFCSHSZARLXAXMXD
ZZEGZHNYZWXXXLFVXAXGRXKRRANFZIKRRZZLLXKEFZXRKGPGRX
DIGXKPXSZGVGFFYVAXXVVXSXKFNKLLVVKAZGGXLPSCPNXLPARG
ESXZFLRAGPXPXRXAPSLFDPFXXXLIXGGAFKXKAYPXPXPX

TABLE 8

The DNA sequence for the human gene for the 24 kD
subunit of Complex I (exon 7) (SEQ ID NO: 7).

ATTAACCCTCACTAAAGGGAACAAAAGCTGGAGCTCCACCGCGGTGGCGC
CGCTCTAGAACTAGTGGGATCCCCCGGGCTGCAGGAAATTCGGCACGAGG
GAANAATCCGNCGCGTCCACAANNACCNTTNNCCCCCAACCAACANNAAN
AACANTTCNNNCNNAAATCNAAGTNCTCCNAGACTNANAATCNNCCATNT
NATNTAAATTTTCNGGGGGGGGGNNCCCNGNAANCNAAATTCCCCCCTTA
NGGAAGGGGGNCCTTNTNNANANGNGNNATNCTTTAAAGNCNAAANGCCT
TTNTNCNNNATAANCCCNTTNTCTTTGGGGGCTCCCNAAATTTTATAACC
NCNAGGANCCNCGGGNTTCTTTNTTTANCNCCCCTTNNAAANTANTTCCC
GGTNTTNAANANCGGNTTCCCCCNCGGTTNTGGGCATNTNTTTTTNCGCG
NCGNTTATAGAGANAAAAAAAAANTTTTNTTCNCCCTTTATACACCGGCA
NTTAAAANTTNGAAAANCNGGGNAANNGGGNGTTTNTTNNAAAAAACNAA
ATNTTTTNTTTNAGCCNCNAAAAAAANCTGAGTTGGCCCCCNCTNNAACC
CCNTTGGNGGGAAAANTNAAAAAGTGCAAACCCCCNCTCTNCCCCNATCT
AGANAAGTAGNNTCCTCCCCCCCTCCCNNAAAANNTAGGGAGNNNCTCCC
GNNNC

TABLE 9

The protein sequence for part of the human gene
for the 24 kD subunit of Complex I (exon 7)
(SEQ ID NO: 14).

LTLTKGNKSWSSTAVAPLNWDPPGCRKFEFPAARGIPLVLERRHRGGAPA
FVPFSEG

TABLE 10

The DNA sequence for the human mitotic
kinesin-like protein-1 (MKLP-1) gene
(SEQ ID NO: 8).

ATTAACCCTCACTAAAGGGAACAAAAGCTGGAGCTCCACCGCGGTGGCGG
CCGCTCTAGAACTAGTGGATCCCCCGGGGNGCNCGAATTCNGAANGAGGC
CTCNTGCCNANTNCTNATGANAGCGAAGGANGTANNNCAGNTCGNACCNG
ATTGACCNTNAGGATATCCANTACNCNANGGGGGGCCCGGNNCCCAATNC
NCCCTATAGTGAGTCNNATCACAATTCACTGGACCGNCGTTTCAAAGGGN
GAGNTTTGGGGGTAAGNCTATACCTAACCCNCTCTCGGNNTTGANTTACA
CGTNCNCGGTCNGTCATTCANCAANCACCAATTGAGTNTTNANCNGGTCC
TCCAGGCTNGNGGTTGCNTNNGGGGGNNCTNAGNANNAAGAATTTTCAAG
GCTGAAATCCCNNTTTAACCCCCAANTNGNNNAGNAAGGGNGGTNCTGCC
CAANNACAAAAAATTTGGGGATANNNGGCAAGGTNANNCCANGTTGNANC
CCAACAGGGTNCCCCCNNGNACAGNAACNTGGGGNNATNTNGAAAACNTC
NNCTNTTNNCNCCCNAATNGNGAGTNAATGGGGGCNNNCCCCCATTTGGN
GAAAAATTNCGNGGANCCGGNCCNCGGGANTTTNAAATNAAANC

TABLE 11

shows the protein sequence for part of mitotic
kinesin-like protein-1 (MKLP-1 gene)
(SEQ ID NO: 9).

INPHZREQKLELHRGGGRSRTSGSPGXXEFXXXRPXAXXXZXRRXXXXXS
XXIDXXDIXYXXGGPXPNXPYSEXXHNSLDXRFKGXXLGVXLYLTXSRXZ
XTRXRXVIXQXPIEXXXGPPGXXLXXGXXXXKNFQGZNPXLTPXXXXKXG
XAQXQKIWGXXARXXXVXXQQGXPXXXNXGXXXKXXXXXPXXXXNGGXPP
FXEKXXGXXXRXFXXKX

TABLE 12

The DNA sequence for exon 2 of human thromboxane
synthase (TBXAS1) (SEQ ID NO: 10).

ATTAACCCTCACTAAAGGGAACAAAAGCTGGAGCTCCACCGCGGTGGCGG
GCCGCTCTAGAACTAGTGGATCCCCCGGGCTGCCCGGTACCCAATTCGCC
CTATAGTGAGTCGTATTACAATTCACTGGCCGTCGTTTTACAAGGGCGAG
CTTGAAGGTAAGCCTATCCCTAACCCTCTCCTCGGTCTCGATTCTACGCG
TACCGGTCATCATCACCATCACCATTGAGTTTAAACGGTCTCCAGCTTGG
CTGTTTTGGCGGATGAGAGAAGATTTTCAGCCTGATACAGATTAAATCAG
AAACGCANGAAGNGGGTCTTGATAAAAACAAGAAATTTGGCCTTGGCGGG
NAGTTAGCNGCGGGTNGGTNCCCACCCTNGACCCCATTGCCCGAAACTCA
CGNAAGNTGAAAACCGCCCGGNAACCGCCCGNATTGGGTAAGTGGTGGGG
GGTCCTTCCCCCATTGCCGAANAAGNTNNGGGGAAACTNGCCCAGGGCAC
TTCAAAATNAAAAAACGNAAAGGGGCTNNANGTCCGAAAAANAAATTGGG
GGCCTTTCCCGGGTTGNAAACCTGGTTGGGTTTGGGGCCGGGGGGAACNC
CTCNTCCTNGNAGTTTNGGACAAAAATCCCGCCNGGGGNNCGCGGGATTT
TGAAACCGTTNTGCNN

TABLE 13

shows the protein sequence for part of Homo
sapiens TBXAS1 gene for thromboxane synthase,
exon 2 (SEQ ID NO: 11).

INPHZREQKLELHRGGGRSRTSGSPGLPGTQFALZZVVLQFTGRRFTRAS
LKVSLSLTLSSVSILRVPVIITITIEFKRSPAWLFWRMREDFQPDTDZIR
NAXXGSZZKQEIWPWRXVSXGXXPTXDPIARNSXKXKTARXPPXLGKWWG
VLPPLPXKXXGNXPRALQNXKTXRGXXSEKXIGGLSRVXNLVGFGAGGNX
XSXXFXTKIPXGXRGILKPXCX

TABLE 14

The DNA sequence for human uncoupling protein
homolog (UCPH) (SEQ ID NO: 12).

TAATACGACTCACTATAGGGCGAATTGGGTACCGGGCCCCCCCTCGAGTT
TTTTTTTTTTTTNNTTNTTTTTTNTTTTNCTNCTTTTNTTTTNTTNTNNN
CTCNCTTTTTCTATNTTCTTTTTNCCTCCACTCTACNGGGGNNTCCCCCG
NGGGGCAAAANCCCNNNNCCNGGGGGNNNCNTNTTTTTTTGGGGGNCCCC
CCCCNGGGGGGGNNCCCNCTTTTTTTTTTCCCTTTNTNTGGGGGGTTTAA
ANGGGGNGNTTNNNGGGGNAGANATTACCNANCCCCCCCCCCCGGNNNCN
NANTTCNCCGCGANTNCCGGNGNGTCTTCCCCCCTTTCCCTTGNGGNTTT
AAAGGGNGCCNCCTNNCTTTCCGNNTTTTTTNNGCNNGGGGAAAAAAAAA
AAAATTTNNCCCCCTGGNTNCCCCCAATTTNANNNCCCCCGNCCCCCCCN
AAANGGTTTTNNNNAAANAAANAAAAANTTTTNCTGGNGGGGGGCNAAAA
AAGNCGGGGGGGGNTCCCCCCCCCGGNNCCCCCTGTGGGGGTAATTTTTC
AAANGGGNNAACCCCCTCNTNTACCCCCNNTTGTTNTCTGGGGGGGGNNC
CCCCCCNCCNCTCCNGAAGAAAGGNGGGATANNGTTCNTCCCTCNACNTA
NAAAAAANN

TABLE 15

shows the protein sequence for part of Human
uncoupling protein homolog (UCPH) mRNA
(SEQ ID NO: 13).

ZYDSLZGELGTGPPLE

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A protein microarray comprising at least a portion of at least two of the following proteins selected from the group consisting of L35 protein, eukaryotic translation elongation factor 1 α-2; NADH dehydrogenase 3 (complex I), 24-kDa subunit of complex I, mitotic kinesin-like protein-1, thromboxane synthase, and uncoupling protein homolog.

2. A protein microarray comprising at least a portion of at least four of the proteins of claim 1.

3. A protein microarray comprising at least a portion of all of the proteins of claim 1.

4. The protein microarray of claim 1 wherein the proteins are His-tagged.

5. The protein microarray of claim 1, wherein the proteins are printed on a charged nickel slide.

6. The protein microarray of claim 1, wherein the L35 protein is represented by a sequence which comprises the sequence shown in SEQ ID NO: 2.

7. The protein microarray of claim 1, wherein the eukaryotic translation elongation factor 1 α-2 is represented by a sequence which comprises the sequence shown in SEQ ID NO: 4.

8. The protein microarray of claim 1, wherein the NADH dehydrogenase 3 (Complex I) protein is represented by a sequence which comprises the sequence shown in SEQ ID NO: 6.

9. The protein microarray of claim 1, wherein the 24-kD subunit of Complex 1 is represented by a sequence which comprises a protein encoded by the sequence shown in SEQ ID NO: 7.

10. The protein microarray of claim 1, wherein the mitotic kinesin-like protein-1 is represented by a sequence which comprises the sequence shown in SEQ ID NO: 9.

11. The protein microarray of claim 1, wherein the thromboxane synthase protein is represented by a sequence which comprises the sequence shown in SEQ ID NO: 11.

12. The protein microarray of claim 1, wherein the uncoupling protein homolog is represented by a sequence which comprises the sequence shown in SEQ ID NO: 13.

13. A method of screening for rheumatoid arthritis in a mammal comprising:
contacting a sample from said mammal to an immobilized polypeptide or fragment thereof homologous to at least a portion of at least one protein selected from the group consisting of L35 protein, eukaryotic translation elongation factor 1 α-2; NADH dehydrogenase 3 (complex I), 24-kDa subunit of complex I, mitotic kinesin-like protein-1, thromboxane synthase, and uncoupling protein homolog; and
detecting binding of an antibody from said sample to said immobilized polypeptide or fragment thereof.

14. The method of claim 13, wherein the polypeptide or fragment thereof is immobilized on a microarray.

15. The method of claim 13, wherein the proteins are His-tagged.

16. The method of claim 13, wherein the proteins are printed on a charged nickel slide.

17. A method of treating rheumatoid arthritis in a mammal comprising administering to said mammal a composition comprising a polypeptide or fragment thereof homologous to at least a portion of at least one protein selected from the group consisting of L35 protein, eukaryotic translation elongation factor 1 α-2; NADH dehydrogenase 3 (complex I), 24-kDa subunit of complex I, mitotic kinesin-like protein-1, thromboxane synthase, and uncoupling protein homolog, said polypeptide or fragment thereof being administered in an amount sufficient to interfere with the binding of an antibody from said mammal.

18. A kit for screening for Rheumatoid Arthritis in a mammal, comprising a mitochondrial marker, homolog or fragment thereof, selected from the group consisting of L35 protein, eukaryotic translation elongation factor 1 α-2, NADH dehydrogenase 3 (complex I), 24-kDa subunit of complex I, mitotic kinesin-like protein-1, thromboxane synthase, and uncoupling protein homolog.

19. The kit of claim 18, wherein said mitochondrial marker, homolog or fragment thereof is immobilized on a rigid white substrate.

20. The kit of claim 18, wherein said mitochondrial marker, homolog or fragment thereof is immobilized on a hydrophobic substrate.
